(19) United States
(12) Patent Application Publication
(10) Pub. No.: US 2024/0341628 A1
(43) Pub. Date: Oct. 17, 2024

(54) SAMPLE COLLECTION DEVICE AND SYSTEM

(71) Applicant: 3M INNOVATIVE PROPERTIES COMPANY, St. Paul, MN (US)

(72) Inventors: Audrey A. Sherman, Woodbury, MN (US); Alan R. Dombrowski, Woodbury, MN (US); Brett J. Sitter, Marine on St Croix, MN (US); Michael R. Berrigan, Oakdale, MN (US); Tonya D. Bonilla, Woodbury, MN (US); Narina Y. Stepanova, Woodbury, MN (US); Laura R. Nereng, Woodbury, MN (US)

(21) Appl. No.: 18/580,292

(22) PCT Filed: Jul. 20, 2022
(86) PCT No.: PCT/IB2022/056714

§ 371 Date: Jan. 18, 2024

Related U.S. Application Data
(60) Provisional application No. 63/306,273, filed on Feb. 3, 2022.
(60) Provisional application No. 63/203,442, filed on Jul. 22, 2021.

(51) Int. Cl. A61B 5/097; A61B 10/00

(52) U.S. Cl. CPC A61B 5/097; A61B 2010/0087

(57) ABSTRACT

A sample collection device includes a housing including an exhalation portion and a coupling portion. The coupling portion can be coupled with/to a sample collection tube. An airflow path extends through the housing. The device further includes porous sample collection media partially disposed between within the housing and arranged to occlude the airflow path. The porous sample collection includes nonwoven material and may include polylactic acid, polypropylene, or a combination thereof. The housing can be a single piece, part, or component, or can be multiple pieces, parts, components, or portions. In an embodiment of a two-piece housing, the first part includes an exhalation piece (e.g., a mouthpiece or nosepiece) and the second part includes a coupling end constructed for coupling with a sample collection tube. A method of obtaining a sample using the sample collection device includes breathing into the exhalation piece or portion to collect a sample on the porous sample collection media; coupling the second part or portion with a sample collection tube; and transferring the collected sample on the porous sample collection media into the sample collection tube.

### FIELD

The present disclosure relates to a sample collection device and system. The present disclosure relates to a bioaerosol collection device and system.

### BACKGROUND

Diagnostic tests used to test for the presence of a virus or other pathogen in the airways, throat, or nasopharynx typically involve the insertion of a swab into the back of the nasal passage, the mid-turbinate area of the nasal passage, the anterior nares, or the throat to obtain a sample. The swab is then inserted into a container and analyzed or sent to a lab for processing. Other diagnostic tests involve collecting a saliva sample and then placing it in a container. Currently available at-home viral tests (e.g., COVID-19 tests) involve a nasal swab and a test kit (for example, the Ellume™ test, the Abbot™ BinaxNOW™ test, and the Lucira™ All-in-One test kit). Tests that utilize nasal swab samples or saliva contend with contaminants that can interfere with the various diagnostic tests. As a result, these sample types require a purification step when using RT-PCR molecular testing.

### SUMMARY

There is a need for an inexpensive, simple to use, and reliable sample collection system that may be used by laypeople to obtain a sample for testing for the presence of a target virus, target pathogen, or other target analyte, in a collected sample. The sample collection system may include a sample collection device for collecting a sample from exhalation airflow onto sample collection media and a transfer mechanism for transferring the sample into a sample collection tube, which may be used to analyze the sample or to transfer the sample to a facility for testing.

It is desirable to provide a sample collection device and system that are easy to use. The device and system may advantageously be self-contained and optionally sterile. A self-contained (and optionally sterile) device and system may improve accuracy and reliability of pathogen testing due to the reduced contamination and background noise, unlike swabs and other test collection devices which may be contaminated upon use and/or during testing.

It is further desirable to provide a system which, after sample collection, provides for easy transfer of the collected sample to a sample collection tube that may be sealed and optionally transferred for testing safely and without contamination.

According to an embodiment, a sample collection device includes a housing including an exhalation portion and a coupling portion. The coupling portion can be coupled with a sample collection tube. An airflow path extends through the housing. The device further includes porous sample collection media partially disposed between within the housing and arranged to occlude the airflow path. The housing can be a single piece, part, or component, or can be multiple pieces, parts, components, or portions.

In one exemplary embodiment, the housing includes a first part and a second part removably coupled with one another, and an airflow path extending through the first and second parts. The first part includes an exhalation piece. The second part includes a coupling end constructed for coupling with a sample collection tube. The device further includes porous sample collection media partially disposed between the first and second parts and arranged to occlude the airflow path. Each of the first part and the second part may have a tubular body. The tubular body of the first part may be coaxial with the tubular body of the second part when the first and second parts are coupled. The second part may further include a coupling mechanism constructed for coupling with a sample collection tube or a cap.

The porous sample collection includes nonwoven material. The nonwoven material may include polylactic acid, polypropylene, or a combination thereof The nonwoven material may be electrostatically charged.

A system for collecting a sample from breath includes the sample collection device and a plunger for releasing the porous sample collection media from the housing. The system may further include a sample collection tube capable of coupling with the second part. The system may be constructed to release the porous sample collection media into the sample collection tube coupled with the sample collection device upon pushing with the plunger.

A method of obtaining a sample using the sample collection device includes breathing into the exhalation piece to collect a sample on the porous sample collection media; coupling the second part with a sample collection tube; and transferring the collected sample on the porous sample collection media into the sample collection tube. The transferring may be done by pushing with a plunger. The transferring may be done by applying a liquid onto the porous sample collection media.

A kit for collecting a sample from breath includes the sample collection device, where the second part has a coupling end constructed for coupling with a sample collection tube, the coupling end having a first size and first coupling configuration; and an adapter constructed to couple with the coupling end of the second part, where the adaptor has a second coupling end having a second size and second coupling configuration that is different from the first size and first coupling configuration.

### BRIEF DESCRIPTION OF FIGURES

FIG. 1A is a side view of a sample collection device according to an embodiment.

FIG. 1B is a perspective view of the sample collection device of FIG. 1A.

FIG. 1C is an exploded view of the sample collection device of FIG. 1A.

FIG. 2A is a side view of a system including the sample collection device of FIG. 1A.

FIG. 2B is a perspective view of the system of FIG. 2A.

FIG. 2C is an exploded view of the system of FIG. 2A.

FIG. 3A is a top view of a first part of the sample collection device of FIG. 1A.

FIG. 3B is a side view of the first part of FIG. 3A.

FIG. 3C is a side cross-sectional view of the first part of FIG. 3A.

FIG. 3D is a top down cross-sectional view of the first part of FIG. 3A.

FIG. 4A is a top view of a second part of the sample collection device of FIG. 1A.

FIG. 4B is a side view of the second part of FIG. 4A.

FIG. 4C is a side cross-sectional view of the second part of FIG. 4A.

FIG. 4D is a bottom up cross-sectional view of the second part of FIG. 4A.

FIG. 5A is a side view of an alternative second part for the sample collection device of FIG. 1A.

FIG. 5B is a bottom perspective view of the alternative second part of FIG. 5A.

FIG. 6A is a side view of another alternative second part for the sample collection device of FIG. 1A.

FIG. 6B is a bottom perspective view of the alternative second part of FIG. 6A.

FIG. 6C is a top perspective view of the alternative second part of FIG. 6A.

FIG. 7 is a partial cross sectional view of the sample collection device of FIG. 1A.

FIG. 8A is a perspective view of the system of FIG. 2A including a plunger.

FIG. 8B is an exploded view of the system of FIG. 8A.

FIG. 9A is a perspective view of an alternative first part for the device of FIG. 1A.

FIG. 9B is a perspective view of the system with the alternative first part of FIG. 9A.

FIG. 10 is a schematic side view of a kit including the sample collection device of FIG. 1A and multiple adapters.

FIG. 11A is a side view of a sample collection device according to an embodiment.

FIG. 11B is a perspective view of the sample collection device of FIG. 11A.

FIG. 12A is a side view of the sample collection device of FIG. 11A.

FIG. 12B is a cross-sectional view of the sample collection device of FIG. 12A.

FIG. 13A is a side view of the first and second parts of the sample collection device of FIG. 11A.

FIG. 13B is a perspective view of the first and second parts of the sample collection device of FIG. 11A.

FIG. 14A is a top view of the first part of the sample collection device of FIG. 11A.

FIG. 14B is a side view of the first part of FIG. 14A.

FIG. 15A is a side view of the first and second parts of the sample collection device of FIG. 11A.

FIG. 15B is a cross-sectional view of the sample collection device of FIG. 14A.

FIG. 16 is a perspective view of a sample collection device according to an embodiment.

FIG. 17 is a perspective view of a system including the sample collection device of FIG. 11A.

FIG. 18 is a partial close-up view of the bottom part of the sample collection device of FIG. 14A.

### DEFINITIONS

All scientific and technical terms used herein have meanings commonly used in the art unless otherwise specified. The definitions provided herein are to facilitate understanding of certain terms used frequently herein and are not meant to limit the scope of the present disclosure.

Unless otherwise indicated, the terms “polymer” and “polymeric material” include, but are not limited to, organic homopolymers, copolymers, such as for example, block, graft, random and alternating
to capture viruses, pathogens, or other analytes, carried in an exhalation copolymers, terpolymers, etc., and blends and modifications thereof Furthermore, unless otherwise specifically limited, the term “polymer” shall include all possible geometrical configurations of the material. These configurations include, but are not limited to, isotactic, syndiotactic, and atactic symmetries.

The terms “downstream” and “upstream” refer to a relative position based on a direction of exhalation airflow through the device. For example, the upstream-most element of the device is the exhalation piece element, and the downstream-most element of the device is the distal end (e.g., the tube coupling end).

All headings provided herein are for the convenience of the reader and should not be used to limit the meaning of any text that follows the heading, unless so specified.

The term “i.e.” is used here as an abbreviation for the Latin phrase id est, and means “that is,” while “e.g.” is used as an abbreviation for the Latin phrase exempli gratia and means “for example.”

All scientific and technical terms used herein have meanings commonly used in the art unless otherwise specified. The definitions provided herein are to facilitate understanding of certain terms used frequently herein and are not meant to limit the scope of the present disclosure.

The term “about” is used here in conjunction with numeric values to include normal variations in measurements as expected by persons skilled in the art and is understood have the same meaning as “approximately” and to cover a typical margin of error, such as ±5% of the stated value. Moreover, unless otherwise indicated, all numbers expressing quantities, and all terms expressing direction/orientation (e.g., vertical, horizontal, parallel, perpendicular, etc.) in the specification and claims are to be understood as being modified in all instances by the term “about.”

Terms such as “a,” “an,” and “the” are not intended to refer to only a singular entity but include the general class of which a specific example may be used for illustration.

The terms “a,” “an,” and “the” are used interchangeably with the term “at least one.” The phrases “at least one of” and “comprises at least one of” followed by a list refers to any one of the items in the list and any combination of two or more items in the list.

As used here, the term “or” is generally employed in its usual sense including “and/or” unless the content clearly dictates otherwise. The term “and/or” means one or all of the listed elements or a combination of any two or more of the listed elements.

The recitations of numerical ranges by endpoints include all numbers subsumed within that range (e.g., 1 to 5 includes 1, 1.5, 2, 2.75, 3, 3.80, 4, 5, etc. or 10 or less includes 10, 9.4, 7.6, 5, 4.3, 2.9, 1.62, 0.3, etc.). Where a range of values is “up to” or “at least” a particular value, that value is included within the range.

As used here, “have”, “having”, “include”, “including”, “comprise”, “comprising” or the like are used in their open-ended sense, and generally mean “including, but not limited to.” It will be understood that “consisting essentially of,” “consisting of,” and the like are subsumed in “comprising” and the like. As used herein, “consisting essentially of” as it relates to a composition, product, method or the like, means that the components of the composition, product, method or the like are limited to the enumerated components and any other components that do not materially affect the basic and novel characteristic(s) of the composition, product, method or the like.

The term “substantially” as used here has the same meaning as “significantly,” and can be understood to modify the term that follows by at least about 90%, at least about 95%, or at least about 98%. The term “not substantially” as used here has the same meaning as “not significantly,” and can be understood to have the inverse meaning of “substantially,” i.e., modifying the term that follows by not more than 10%, not more than 5%, or not more than 2%.

The words “preferred” and “preferably” refer to embodiments that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the disclosure, including the claims.

Any direction referred to here, such as “front,” “back,” “top,” “bottom,” “left,” “right,” “upper,” “lower,” and other directions and orientations are described herein for clarity in reference to the FIGS. and are not to be limiting of an actual device or system or use of the device or system. Devices or systems as described herein may be used in a number of directions and orientations.

Any direction referred to here, such as “top,” “bottom,” “left,” “right,” “upper,” “lower,” and other directions and orientations are described herein for clarity in reference to the figures and are not to be limiting of an actual device or system or use of the device or system. Devices or systems as described herein may be used in a number of directions and orientations.

### DETAILED DESCRIPTION

The present disclosure relates to a sample collection device and system. The present disclosure relates to a bioaerosol sample collection device and system.

The sample collection device includes porous sample collection media along an airflow path defined by the device housing. The porous sample collection media is constructed airflow. After loading, the sample collection media may be transferred into a sample collection tube. The collection media may be transferred into a sample collection tube The sample may further be analyzed for the presence of a pathogen of interest, or the sample collection tube may be capped and sent away for analysis. According to an alternative embodiment, the sample may be transferred into the sample collection tube by passing a liquid through the porous sample collection media to elute the sample, including pathogens, viruses, or other analytes, bound to the porous sample collection media, forming an eluate, and allowing the eluate to flow into the sample collection tube. The eluate may then be analyzed using known methods.

According to an embodiment, the sample collection device includes a housing. The housing includes a first part and a second part that define an airflow path. Porous sample collection media is disposed within the housing and arranged to occlude the airflow path. The porous sample collection media may be captured between the first and second parts. According to an embodiment, the porous sample collection media is captured between the first and second parts in such a way that it stays in place during sample loading but can be easily pushed out when desired. The user may exhale into the sample collection device and load the porous sample collection media with a sample of the exhalation airflow to form a loaded porous sample collection media. The user may exhale through the opening in an exhalation piece on the first part. The exhalation piece may be used for exhalation through the mouth (e.g., may be a mouthpiece) or through the nose (e.g., may be a nosepiece). The housing is constructed so that by exhaling through the opening in the exhalation piece, the exhalation airflow passes through the porous sample collection media. The porous sample collection media is constructed to capture viruses, other pathogens, or other analytes, from the exhalation airflow. The user may then transfer the loaded sample collection media into a sample collection tube. Alternatively, as noted above, the user may elute the sample from the loaded sample collection media directly into the sample collection tube.

The first and second parts of the housing are removably coupled with one another. In an alternative embodiment, the first and second parts are permanently coupled with one another such that they parts cannot be separated without breaking or deforming. The first piece has a proximal end and an opposite distal end. The proximal end may form an exhalation piece, such as a mouthpiece or a nosepiece. The exhalation piece may be configured to accommodate exhalation through either the mouth or the nose. In some embodiments, the distal end of the first part is received in the second part. This way, the second part is the outer part and the first part is the inner part. Alternatively, the device may be configured such that the second part is received in the first part, where the first part is the outer part and the second part is the inner part, and the features connecting the first and second parts are reversed. The second part (or outer part) may include a ledge on the interior of the second part. The outer edge of the sample collection media may be captured between the ledge of the second part and the distal end of the first part.

According to an embodiment, the housing has a longitudinal center axis. The airflow channel extends through both the first and the second part. The airflow channel may extend along the longitudinal center axis. One or both of the first and second parts may include a tubular body. When the first and second parts are coupled, the tubular bodies may be coaxial.

The first and second parts may be coupled with any suitable mechanism. For example, the first and second parts may be coupled by bayonet coupling, interference fit, snap fit, or threaded coupling. In one embodiment, the first and second parts are coupled by bayonet coupling. When configured for bayonet coupling, the first part may include one or more protrusions and the second part may include one or more corresponding grooves constructed to receive and guide the one or more protrusions. Alternatively, the one or more protrusions may be on the second part and the one or more grooves may be on the first part.

According to an embodiment, the second part has a proximal end and an opposing distal end. The proximal end of the second part is constructed to couple with the first part (e.g., constructed to receive the distal end of the first part). The distal end of the second part is a tube coupling end that is constructed to couple with a sample collection tube. The tube coupling end may include any suitable mechanism for coupling with a sample collection tube. For example, the tube coupling end may by constructed for bayonet coupling, interference fit, snap fit, or threaded coupling. Many commercially available sample collection tubes or test tubes have a threaded top for attaching a cap. The tube coupling end of the second part may be constructed to couple with the threads of the sample collection tube. The tube coupling end of the second part may include internal threading configured to couple with the external threading of the sample collection tube. The tube coupling end of the second part may include two or more different threads having different configurations (e.g., size, spacing of threads, or angle of threads) to provide attachment for different types or sizes of the sample collection tubes. The device may also be provided with adapters for changing the size or type of coupling to facilitate different types or sizes of sample collection tubes.

In some embodiments, the tube coupling end of the second part is configured for an interference fit with a sample collection tube. To facilitate an interference fit, the tube coupling end may include a protrusion sized to be received inside a sample collection tube. The coupling end may include a seal, such as an O-ring, to seal the tube coupling end against the sample collection tube.

To further facilitate holding the sample collection tube in place by interference fit, the device May include a finger support. A user may hold the sample collection tube in place by holding the end of the tube with one finger and the finger support with another finger. The finger support may include one or more extensions or flanges extending from the first part or the second part.

The loaded sample collection media may be transferred into the sample collection tube by pushing it into the tube with a plunger while the sample collection tube is coupled with the sample collection device. In some embodiments, the plunger is a rod configured to fit through the airflow channel The plunger may include a first end configured as a finger grip and an opposing second end configured to contact and push and dislodge the sample collection media. In some embodiments, the plunger is constructed to remain inside the device after transferring the sample collection media. The plunger may be disposed of with the device.

The porous sample collection media may be a nonwoven material capable of capturing pathogens, viruses, or other analytes from an exhalation airflow. According to an embodiment, the porous sample collection media is a nonwoven material carrying an electrostatic charge. The electrostatic charge may enable capturing pathogens, viruses, or other analytes from an exhalation airflow. In some cases, the porous sample collection media may be a hydrophobic nonwoven material. In other cases, the porous sample collection media may be a hydrophilic nonwoven material. The porous sample collection media may be a hydrophobic nonwoven material carrying an electrostatic charge configured to capture pathogens, viruses, or other analytes from an exhalation airflow. The porous sample collection media may be a hydrophilic nonwoven material carrying an electrostatic charge configured to capture pathogens, viruses, or other analytes from an exhalation airflow. The term “hydrophobic” refers to a material having a water contact angle of 90 degrees or greater, or from about 90 degrees to about 170 degrees, or from about 100 degrees to about 150 degrees. The term “hydrophilic” refers to a material having a water contact angle of less than 90 degrees. Water contact angle is measured using ASTM D5727-1997 Standard test method for surface wettability and absorbency of sheeted material using an automated contact angle tester.

The porous sample collection media may be formed of any suitable material that is capable of capturing viruses, pathogens, or other analytes from exhalation airflow and releasing the captured viruses, pathogens, or other analytes upon being contacted with an eluent, such as a saline solution. The porous sample collection media may be formed of polymeric material. The porous sample collection media may be formed of a polyolefin. Examples of suitable polyolefins include polypropylene, polylactic acid, and the like, and a combination thereof. In one embodiment the porous sample collection media is formed of polypropylene. In one embodiment the porous sample collection media is formed of polylactic acid. One illustrative porous sample collection media is commercially available from 3M Company (St. Paul MN, U.S.A.) under the trade designation FILTRETE Smart MPR 1900 Premium Allergen, Bacteria & Virus Air Filter Mery 13.

The porous sample collection media may have a thickness (orthogonal to the major plane) of 200 82 m or greater or 250 μm or greater. The porous sample collection media may have a thickness of 750 μm 15 or less or 1000 μm or less. The porous sample collection media may have a thickness of in a range from 200 μm to 1000 μm, or from 250 μm to 750 μm. The porous sample collection media may have major plane surface area (of one side) of 1 cm² or greater or 2 cm² or greater. The porous sample collection media may have major plane surface area of 3 cm² or less or 4 cm² or less. The porous sample collection media may have major plane surface area in a range from 1 cm² to 4 cm², or 2 cm² to 3 cm².

The housing may be formed of any suitable material. The housing may be formed of a rigid material, such as plastic, metal, glass, or the like, or a combination thereof.

The sample collection system may be provided as a kit. The kit may include the sample collection device as discussed above and one or more adapters configured to couple various sizes or styles of sample collection tubes to the device.

In some embodiments, the sample is eluted from the sample collection media by dispensing a liquid onto the loaded sample collection media. The liquid dispensed onto the sample collection media may be an aqueous liquid. The liquid may be a buffer solution. The liquid may be an aqueous buffer solution. The liquid may be a saline solution. The liquid may include a surfactant. The liquid may have a contact angle of greater than 90 degrees when measured on the porous sample collection media. The liquid may be a saline solution including a surfactant. The liquid (e.g., a buffer or a saline solution) may include from 0.1 wt-% or more or 0.5 wt-% or more, and up to 1 wt-% or up to 2 wt-% of surfactant. When provided as a metered dose, the liquid may have a volume of 50 μL to 500 μL.

The liquid may be applied onto the loaded porous sample collection media. The liquid may travel through the surface and thickness of the loaded porous sample collection media and flow off of the porous sample collection media carrying any virus, pathogen, or other analyte, that was present on the loaded porous sample collection media. This loaded liquid may then be collected and tested, as described herein.

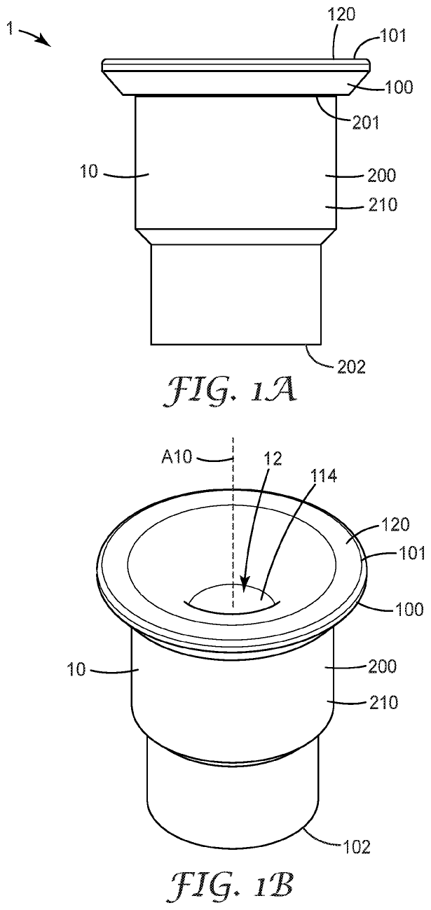

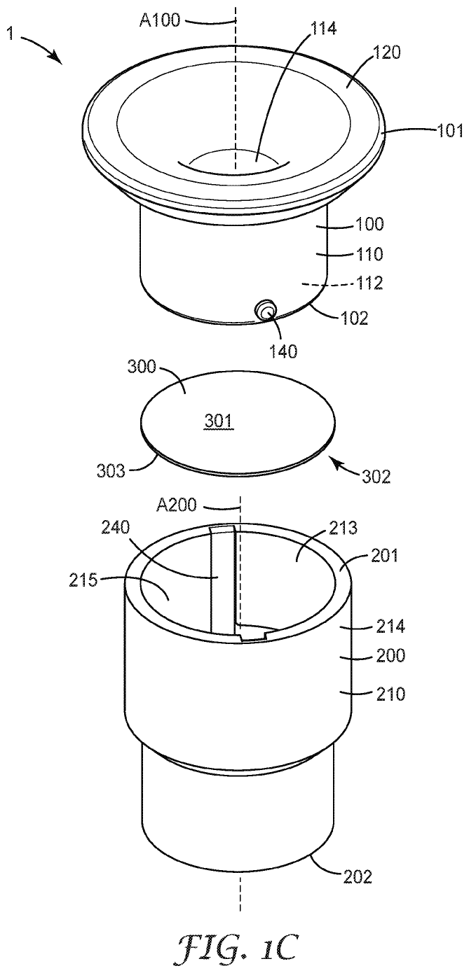

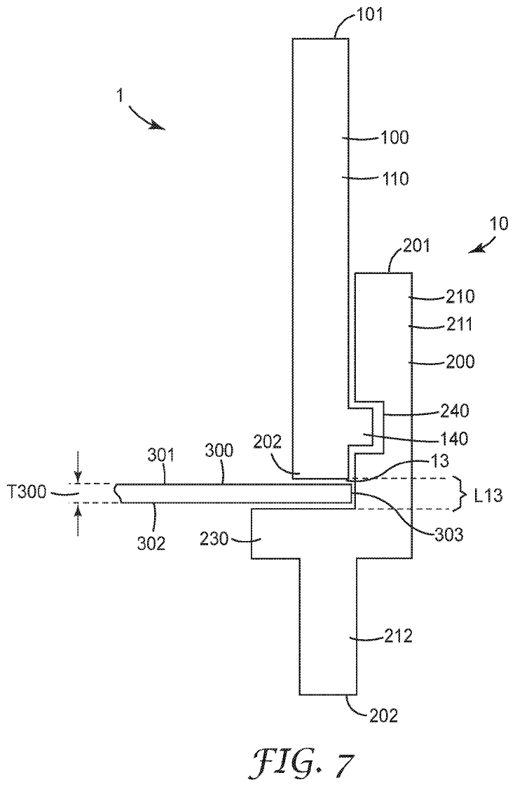

Referring now to FIGS. 1A-1C, a sample collection device 1 is shown. The sample collection device 1 has a housing 10 made up of two parts, a first part 100 and a second part 200. The first and second parts 100, 200 are coupled together to form a breath tube with an airflow path 12 extending therethrough. As further explained below, between the first and second parts 100, 200 there is formed a gap 13 (see FIG. 7). A piece of porous sample collection media 300 is disposed within the housing 10 such that the porous sample collection media 300 occludes the airflow path 12. The outer edge 303 of the porous sample collection media 300 may be captured in the gap 13 between the first and second parts 100, 200.

The first part 100 is configured for coupling with the second part 200. In the embodiment shown, the distal end 102 of the first part 100 is received in the second part 200. The outside surface 112 of the tubular body 110 may include a coupling mechanism. A corresponding coupling mechanism may be formed on the inside surface of the second part 200. In the embodiment shown, the coupling mechanism is a bayonet connection, although other coupling mechanisms may also be used. The outside surface 112 of the tubular body 110 of the first part 100 includes one or more (e.g., two) protrusions 140. The one or more protrusions 140 may be received in one or more corresponding grooves 240 on the inside surface 213 of the second part 200.

The housing 10 has a longitudinal center axis A10. In the embodiment shown, the airflow path 12 extends along the longitudinal center axis A10 of the housing 10. The airflow path 12 extends through both the first part 100 and the second part 200. The first and second parts 100, 200 may be coaxial when coupled together.

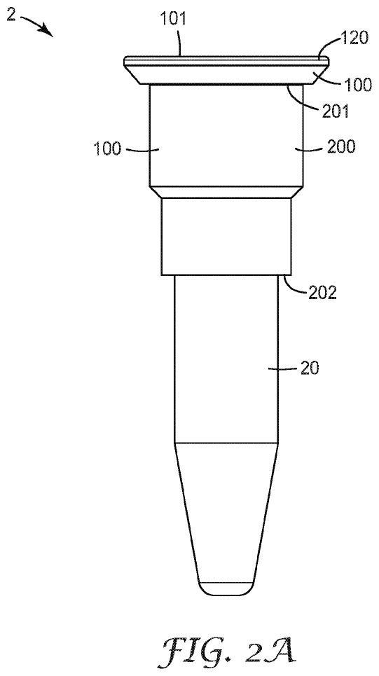

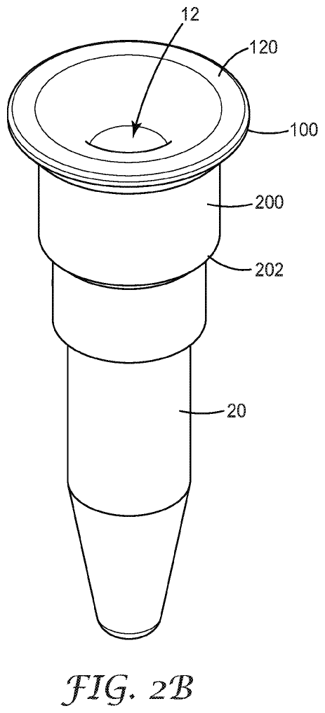

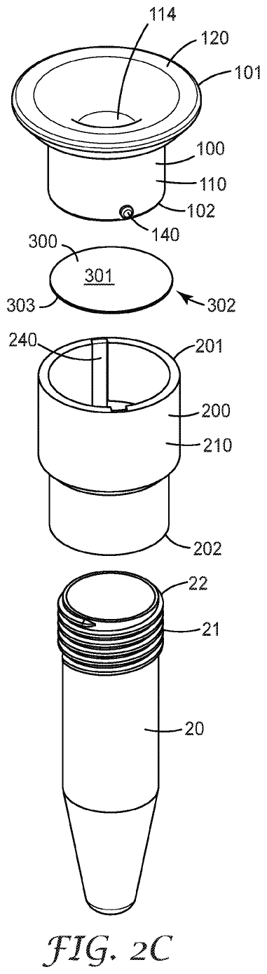

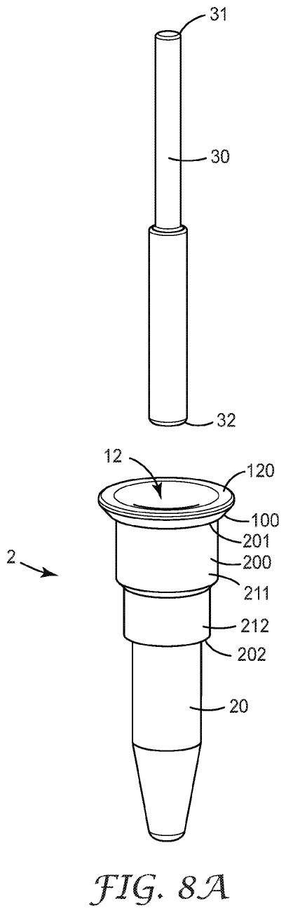

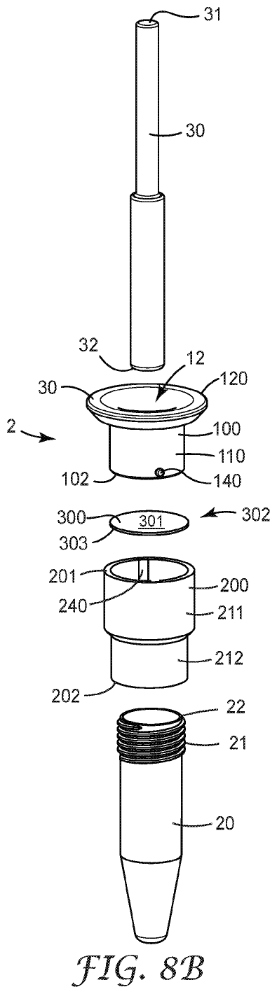

According to an embodiment, the sample collection device 1 may be coupled with a sample collection tube 20, as shown in FIGS. 2A-2C. The second part 200 has a distal end 202 configured as a tube coupling end for coupling with the sample collection tube 20. After loading a sample onto the porous sample collection media 300 by breathing into the sample collection device 1, a user may transfer the sample into the sample collection tube 20. The sample may be transferred by transferring the loaded sample collection media 300 into the sample collection tube 20, for example, by dislodging the sample collection media 300 using a plunger 30 (see FIGS. 8A and 8B). Alternatively, the sample may be transferred by eluting the sample with a suitable eluent, as discussed above. The eluent may be applied through the airflow path 12.

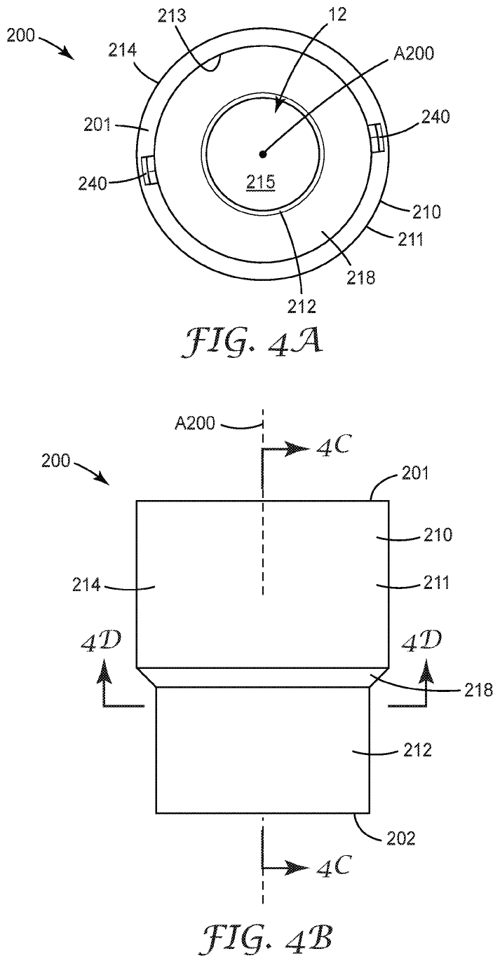

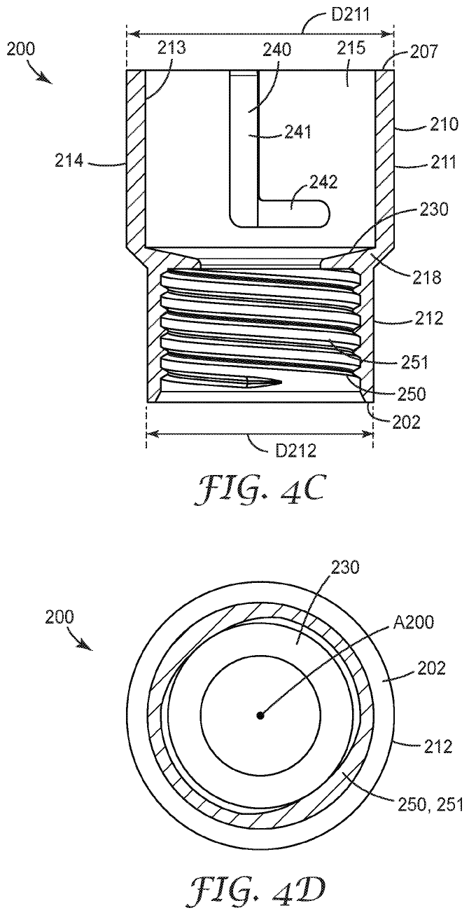

The second part 200 may include a tube coupling mechanism 250 adjacent the distal end 202 (see FIGS. 4A to 6C). In the embodiment shown, the tube coupling mechanism 250 comprises threads 251. A sample collection tube 20 with a threaded mouth 22 may be coupled with the threads 251. The sample collection tube 20 may include external threads 21.

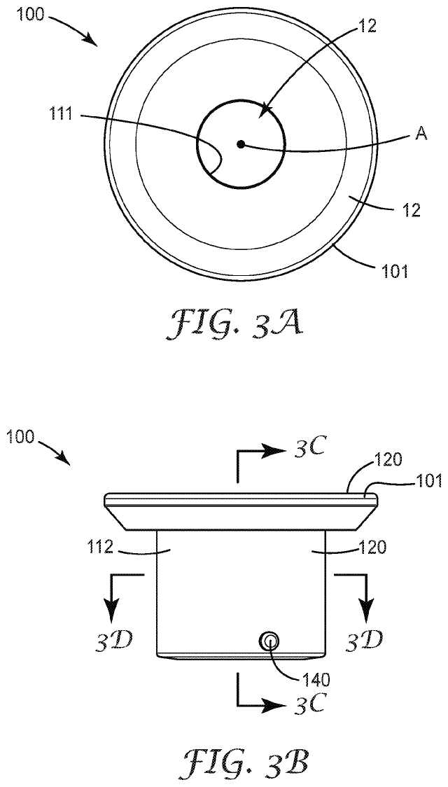

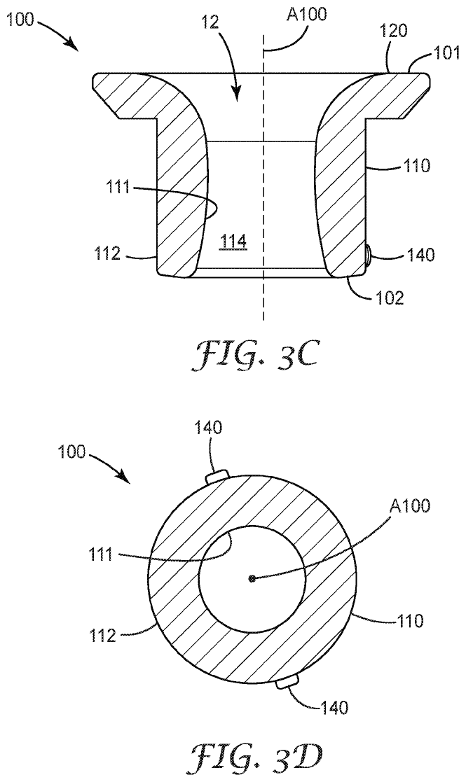

Referring now to FIGS. 3A-3D, the first part 100 of the housing 10 is shown in more detail. The first part 100 defines a proximal end 101 and an opposing distal end 102. The airflow path 12 extends through the first part 100 from the proximal end 101 to the distal end 102. The airflow path 12 may extend along a longitudinal center axis A100 of the first part 100. The first part 100 may have a tubular body 110 defining an inside surface 111, outside surface 112, and an interior 114. The interior 114 forms a portion of the airflow path 12. An exhalation piece 120 may be formed at the proximal end 101. The exhalation piece 120 may be a ring extending outwardly from the tubular body 110, as shown. The protrusions 140 extend outwardly from the outside surface 112 of the tubular body 110.

Referring now to FIGS. 4A-4D, the second part 200 of the housing 10 is shown. The second part 200 defines a proximal end 201 and an opposing distal end 202. The airflow path 12 extends through the second part 200 from the proximal end 201 to the distal end 202. The airflow path 12 may extend along a longitudinal center axis A200 of the second part 200. The second part 200 may have a tubular body 210 defining an interior 214. The interior 214 forms a portion of the airflow path 12. The interior 214 at the proximal end 201 may be configured for receiving and coupling with the first part 100. The interior 214 at the proximal end 201 may include a coupling mechanism configured to cooperate with the coupling mechanism on the first part 100. In the embodiment shown, the second part 200 has one or more (e.g., two) grooves 240 constructed to receive the one or more protrusions 140 of the first part. The grooves 240 may include a longitudinal part 241 and a connected horizontal part 242. The longitudinal part 241 may extend parallel to the longitudinal center axis A200.

The distal end 202 of the second part 200 may be configured as the tube coupling end. The second part 200 may include a tube coupling mechanism 250 adjacent the distal end 202. In the embodiment shown, the tube coupling mechanism 250 includes threads 251. However, other coupling mechanisms may also be used. The threads 251 are formed on the inside surface 213 of the second part 200. A sample collection tube 20 with a threaded mouth 22 may be coupled with the threads 251 (see FIG. 2C).

The diameter of the second part 200 may vary along the length of the tubular body 210. For example, as shown, the proximal portion 211 that includes the coupling mechanism for the first portion 100 may have a diameter D211 that is different from the diameter D212 of the distal portion 212 that includes the coupling mechanism for the sample collection tube 20. Both the inside and outside diameters may be selected independently.

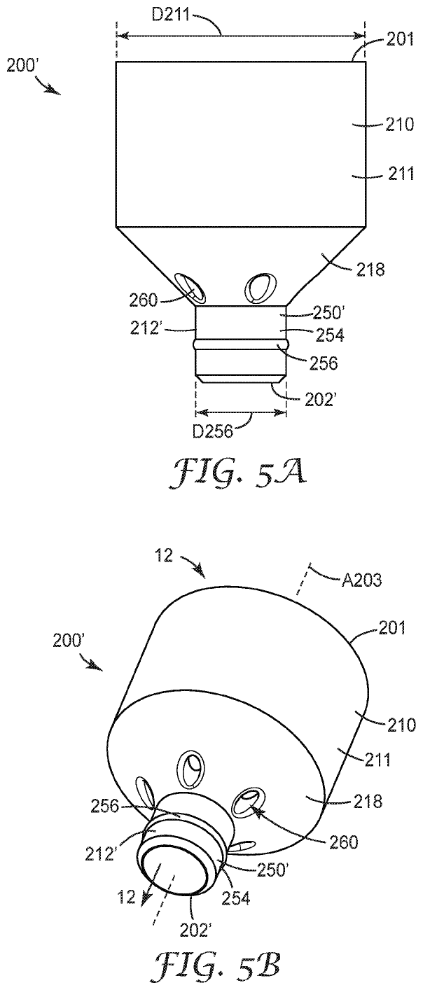

In some embodiments, the second part has an alternative coupling mechanism, such as an interference fit mechanism. An example of a second part 200′ configured for interference fit with a sample collection tube 20 is shown in FIGS. 5A and 5B. The second part 200′ may be otherwise constructed similar to the second part 200 shown, for example, in FIGS. 4A-4D. The coupling mechanism 250′ at the distal end 202′ of the second part 200′ includes an extension 254 configured to be received inside a sample collection tube 20. The extension 254 may also have a seal 256 (such as an O-ring) to improve the fit with various sample collection tubes 20. The seal 256 may generate a seal between the extension 254 and an inside surface of the sample collection tube 20. The second part 200′ may include a diameter reducing portion 218 that reduces the diameter from the diameter D211 of the proximal portion 211 to the diameter D254 of the extension 254. The diameter reducing portion 218 may be conical, as shown. A conical surface may also act as a coupling surface with the sample collection tube 20.

The airflow path 12 extends through the second part 200′ and through the extension 254. The distal portion 212′ of the second part 200′ may optionally include air bypass holes 260. This may facilitate easier blowing of air through the device if the extension 254 and consequently the airflow path 12 are narrow and restrict airflow.

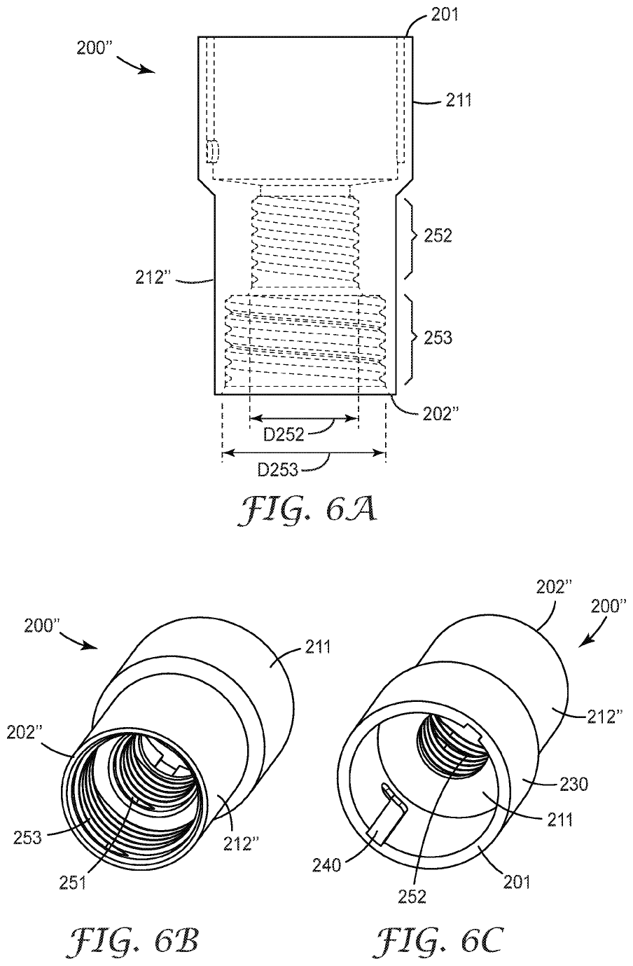

Another alternative tube coupling mechanism is shown in FIGS. 6A-6C. To facilitate sample collection tubes 20 of different sizes (e.g., having different diameters), the distal portion 212″ of the second part 200″ may include multiple sets of threads 252, 253. The second part 200″ (including the proximal portion 211) may be otherwise constructed similar to the second part 200 shown in FIGS. 4A-4D. A first set of threads 252 may have a first diameter D252 and a second set of threads 253 May have a second diameter D253 that is larger than the first diameter D252. The second set of threads 253 is distal to the first set of threads 252. A smaller diameter sample collection tube may be pushed further into the second part 200″ to reach the smaller first set of threads 252, whereas a larger diameter sample collection tube may be threaded onto the larger second set of threads 253.

Referring now to FIG. 7, a schematic partial cross-sectional view of the device 1 is shown. According to an embodiment, the porous sample collection media 300 is captured between the distal end 102 of the first part 100 and the ledge 230 of the second part. When the first part 100 and the second part 200 are coupled, for example by a bayonet connection, a gap 13 is formed between the distal end 102 of the first part 100 and the ledge 230 of the second part. The gap 13 has a length L13. The porous sample collection media 300 has a thickness T300 that is equal to or smaller than the length L13 of the gap 13. In some embodiments, the thickness T300 is smaller than the length L13. This enables the porous sample collection media 300 to be easily dislodged when desired.

The sample collection media 300 may be disposed inside the housing 10 such that first and second major surfaces 301, 302 of the sample collection media 300 are perpendicular or substantially perpendicular to the airflow path 12. The sample collection media 300 is preferably sized and shaped such that the outer edge 303 of the sample collection media 300 is captured in the gap 13 along the entire perimeter of the sample collection media 300. While the porous sample collection media 300 is illustrated here as defining a substantially round planar element, it is understood that the porous sample collection media may define any shape when disposed within the housing and along the airflow path. The shape and size of the porous sample collection media 300 may be selected based on the shape and size of the interiors 114, 215 of the first and second parts 100, 200.

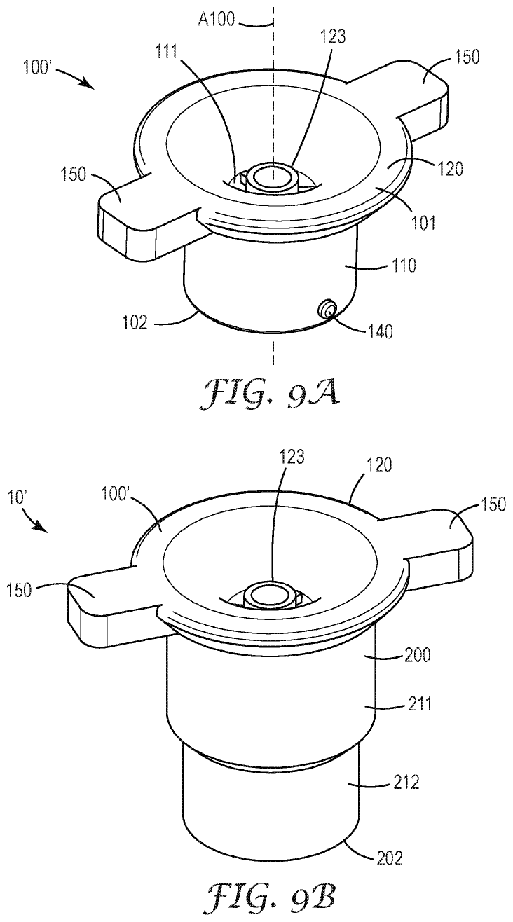

The sample collection device 1 may be provided as a system 2 that also includes a plunger 30 configured to dislodge the porous sample collection media 300 from the device 1, as shown in FIGS. 8A and 8B. According to an embodiment, the housing 10 may be coupled with a sample collection tube 20 and the plunger 30 may be used to push through the housing 10 to press the porous sample collection media 300 down into the sample collection tube 20. The plunger 30 may be a rod that is sized to fit through the exhalation piece 120 and the airflow path 12. In some embodiments, the first part 100 may include a plunger guide 123, as shown in FIGS. 9A and 9B. The plunger 30 may include a first end 31 configured as a finger grip and an opposing second end 32 configured to contact and dislodge the sample collection media 300.

The plunger guide 123 may help guide the plunger 30 through the middle of the housing 10. This may be desirable, in particular, in embodiments where the sample collection tube 20 has a larger diameter. The plunger guide 123 includes a tubular element attached to the inside surface 111 the first part 100. The plunger guide 123 is centered along the longitudinal center axis A100 of the first part.

The housing 10′ may include one or more finger supports 150, as shown in FIGS. 9A and 9B. A user may hold the sample collection tube 20 in place against the housing 10′ by holding the distal end of the tube 20 with one finger and the finger supports 150 with one or more other fingers. The finger supports 150 include extensions extending from the outside surface 112 of the first part 100′. Alternatively, the finger supports 150 may extend from the second part 200.

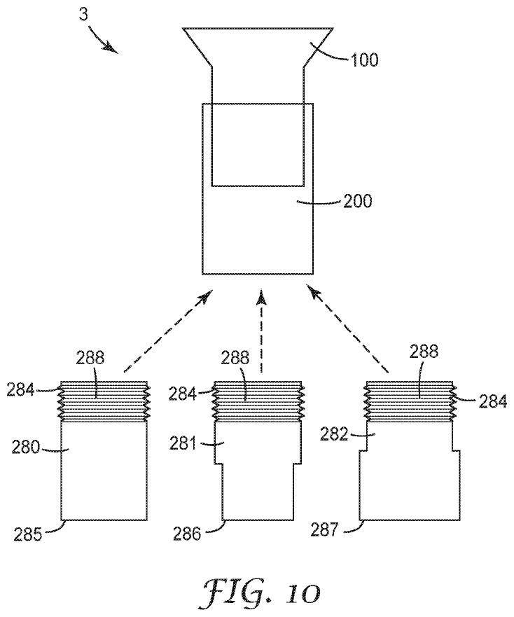

In some embodiments, the sample collection device 1 is provided as a kit 3 that includes the sample collection device 1 and one or more adapters 280, 281, 282, as shown in FIG. 10. The adapters 280, 281, 282 are configured to couple various sizes or styles of sample collection tubes to the device 1. Each adapter 280, 281, 282 has a first end 284 that is configured to couple with the tube coupling mechanism 250 of the second part 200. The first end 284 may have threads 288 to couple with corresponding threads 251 on the second part 200. Alternatively, the first end 284 may be a simple (smooth) tube for an interference with the second part 200, for example the second part 200′ shown in FIGS. 5A and 5B. Each adapter 280, 281, 282 has a second end 285, 286, 287, configured to couple with a sample collection tube 20. The kit 3 may include multiple different size adapters 280, 281, 282, with varying size second ends 285, 286, 287. For example, one adapter 280 may have a second end 285 with narrow diameter threading. A second adapter 281 may have a second end 286 with medium diameter threading. A third adapter 282 may have a second end 287 with wide diameter threading. The threading at the second ends 285, 286, 287 may be internal threads, similar to the threads 251 on the second part 200. One or more of the adapters 280, 281, 282 may alternatively be configured for interference fit with the sample collection tube 20.

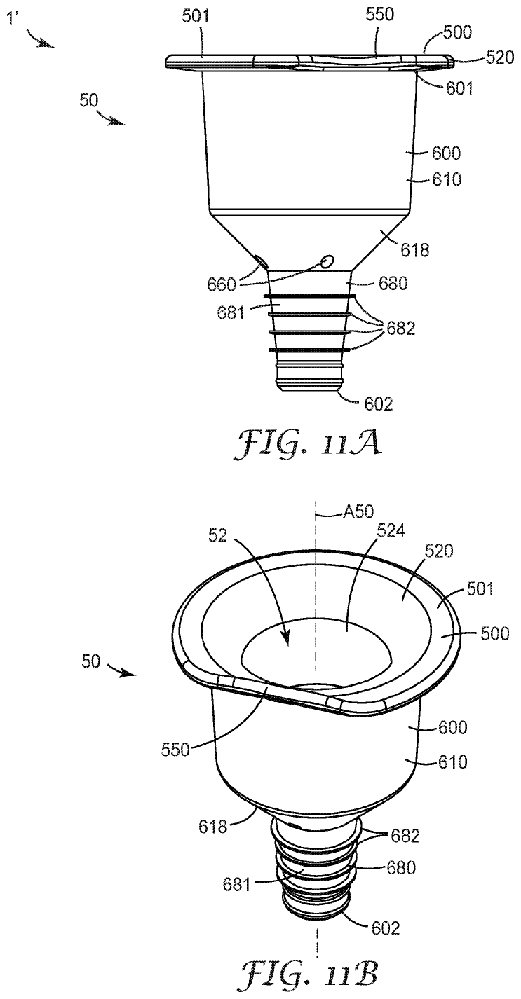

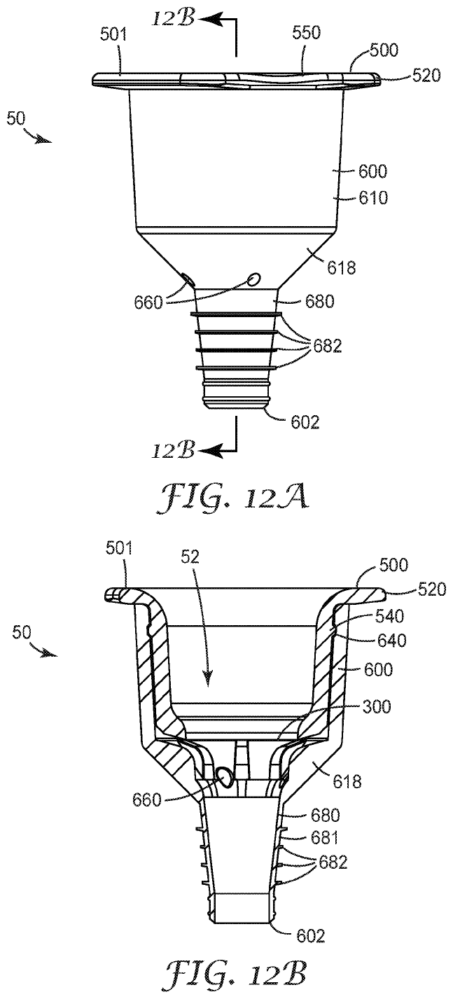

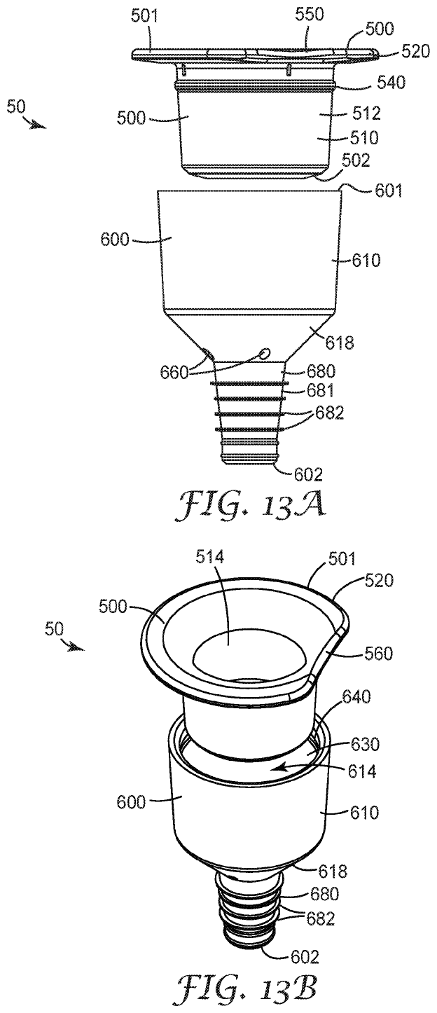

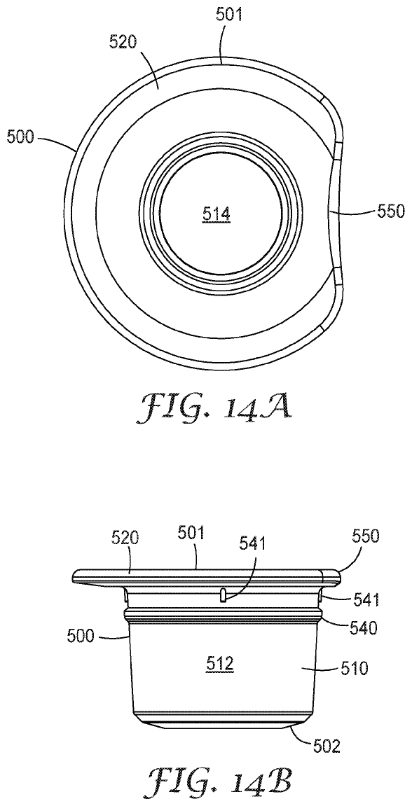

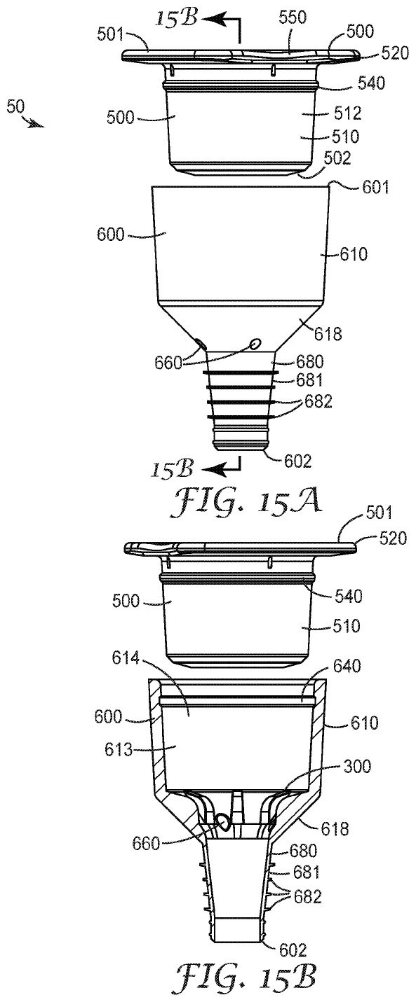

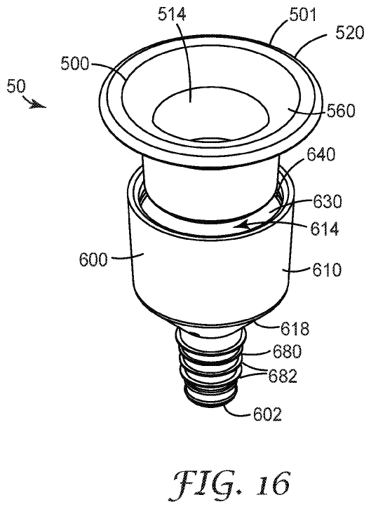

An alternative embodiment of a sample collection device 1′ is shown in FIGS. 11A-18. The sample collection device 1′ has a housing 50 made up of two parts, a first part 500 and a second part 600. The first and second parts 500, 600 are coupled together to form a breath tube with an airflow path 52 extending therethrough. A piece of porous sample collection media 300 is disposed within the housing 50 such that the porous sample collection media 300 occludes the airflow path 52, similar to the sample collection device 1.

The first part 500 is configured for coupling with the second part 600. The first part 500 defines a proximal end 501 and an opposing distal end 502. The first part 500 may have a tubular body 510 defining an interior 514 and an outside surface 512. The interior 514 forms a portion of the airflow path 52. A flange 520 may be formed at the proximal end 501 of the tubular body 510. The flange 520 may extend outwardly from the proximal end 501. The flange 520 may act as a mouthpiece or a nosepiece. The distal end 502 of the first part 500 is received in the second part 600.

The second part 600 defines a proximal end 601 and an opposing distal end 602. The airflow path 52 extends through the second part 600 from the proximal end 601 to the distal end 602. The second part 600 may have a tubular body 610 defining an interior 614. The interior 614 at the proximal end 601 may be configured for receiving and coupling with the first part 500. The outside surface 512 of the tubular body 510 may include a coupling mechanism. A corresponding coupling mechanism may be formed on the inside surface 613 of the second part 600. In the embodiment shown, the coupling mechanism is a snap connection, although other coupling mechanisms may also be used. The outside surface 512 of the tubular body 510 of the first part 500 includes a ring 540. The ring 540 may be received in a corresponding groove 640 on the inside surface 613 of the second part 600. The outside surface 512 of the tubular body 510 may include protrusions 541. The protrusions 541 may help align the first and second parts 500, 600. The protrusions 541 may help maintain the orientation of the first and second parts 500, 600 along a vertical axis, or when disposed at a fixed or variable angle relative to one another.

The housing 50 has a longitudinal center axis A50. In the embodiment shown, the airflow path 52 extends along the longitudinal center axis A50 of the housing 50. The airflow path 52 extends through both the first part 500 and the second part 600. The first and second parts 500, 600 may be coaxial when coupled together.

The second part 600 may include a diameter reducing portion 618, similar to the diameter reducing portion 218 of the second part 200. The second part 600 may include air bypass holes 660. The air bypass holes 660 may be positioned in the diameter reducing portion 618.

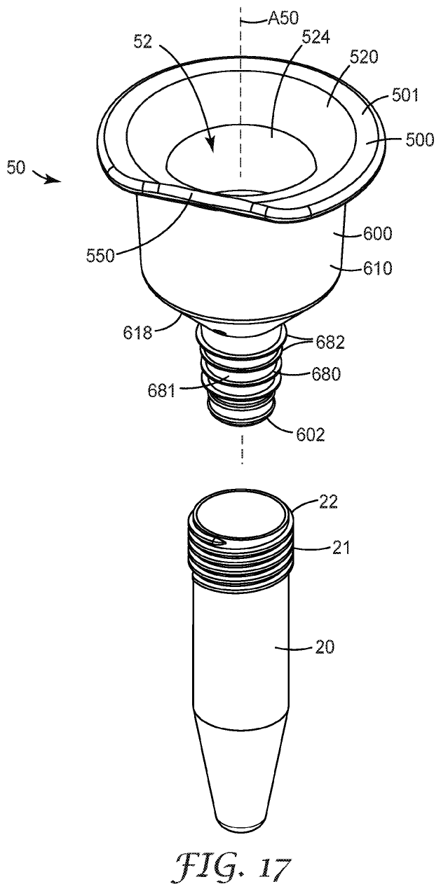

According to an embodiment, the sample collection device 1′ may be coupled with a sample collection tube 20, as shown in FIG. 17. The second part 600 has a distal end 602 configured as a tube coupling end for coupling with the sample collection tube 20. After loading a sample onto the porous sample collection media 300 by breathing into the sample collection device 1′, a user may transfer the sample into the sample collection tube 20. The sample may be transferred by transferring the loaded sample collection media 300 into the sample collection tube 20, for example, by dislodging the sample collection media 300 using a plunger 30 (see FIGS. 8A and 8B). Alternatively, the sample may be transferred by eluting the sample with a suitable eluent, as discussed above. The eluent may be applied through the airflow path 52.

The second part 600 includes a tube coupling extension 680 adjacent the distal end 602. The tube coupling extension 680 may include features that help secure the tube coupling extension 680 inside a sample collection tube 20. In the embodiment shown, the tube coupling extension 680 includes a frustoconical portion 681 and one or more rings or ribs 682 extending outwardly from the frustoconical portion 681. The frustoconical portion 681 may be tapered toward the distal end 602, as shown. According to an embodiment, the tube coupling extension 680 includes a plurality of ribs 682. The ribs 682 engage at least partially with the inside surface of the sample collection tube 20. The ribs 682 may help hold the sample collection device 1′ connected to the sample collection tube 20 while a sample is transferred into the sample collection tube 20.

According to an embodiment, the ribs 682 may be deformable. That is, when the tube coupling extension 680 is inserted into a sample collection tube 20, the ribs 682 are deformed past their yield point to provide the interference fit to the sample collection tube 20. The frustoconical portion 681 may also be formed by a thin wall that provides additional flexibility to help with the fitment.

The plurality of ribs 682 may have different sizes or diameters. The size of the ribs 682 may gradually increase, beginning with the smallest diameter adjacent the distal end 602 and ending with the largest diameter adjacent the diameter reducing portion 618. That is, the plurality of ribs 682 may include at least a first rib adjacent the distal end 602 having a first diameter, and a second rib further away from the distal end 602 and having a second diameter that is greater than the first diameter. The plurality of ribs 682 may further include additional ribs with progressively increasing diameters. The different sizes of the ribs 682 and the tapered frustoconical portion 681 allow connection to sample collection tubes 20 of different inside diameters without concern about the specific dimensions of threads or outside diameter of the tube.

In the embodiment shown, the ribs 682 are radial ribs that extend circumferentially around the frustoconical portion 681. However, in some embodiments the ribs 682 have a different shape that facilitates a mechanical interference fit with the inside of a sample collection tube. For example, the ribs 682 may extend only partially around the frustoconical portion 681, or may form fins extending axially along the frustoconical portion 681, or may form a wedge.

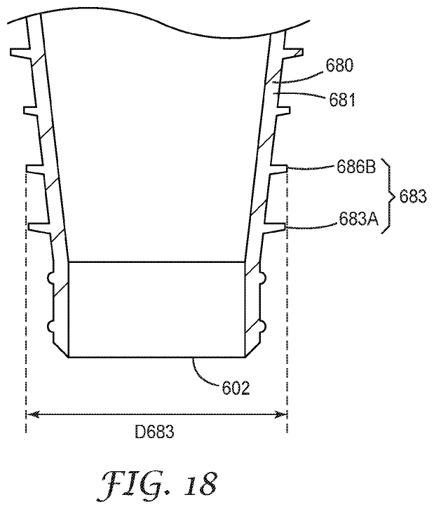

In some embodiments, the tube coupling extension 680 includes one or more pairs of ribs 683, where the ribs of a pair of ribs have the same diameter, as shown in FIG. 18. That is, the tube coupling extension 680 may include a first pair of ribs 683 having two ribs 683A, 683B that each extend to the same (first) outer diameter D683. Alternatively, instead of a pair of ribs, the tube coupling extension 680 may include a group of ribs that includes three or more ribs of the same diameter. The tube coupling extension 680 may further include a second pair of ribs having two ribs that each extend to the same (second) diameter. The first pair of ribs 683 may be closest to the distal end 602. The first diameter D683 may be smaller than the second diameter. The tube coupling extension 680 may further include additional pairs of ribs, each pair having successively greater diameters. Having a pair (or group) of ribs with the same diameter may prevent or reduce rocking or pivoting of the sample collection device 1, 1′ when the tube coupling extension 680 is inserted in the sample collection tube 20.

In some embodiments, the housing 50 (e.g., the first part 500) is modified to accommodate sampling from nasal breath or aerosol alone or in addition to mouth breath or aerosol. The flange 520 of the first part 500 may include a cut-away 550, as shown, for example, in FIGS. 11B, 13B, 14A, and 14B. The cut-away 550 may have a curved edge that may comfortably be placed against the base of the nose of a user. Placing the cut-away 550 the base of the nose may help align the airflow path 52 with the rim of the nostril.

The sample collection system may further comprise a machine-readable optical label. Such labels may include, for example, a bar code and a QR (quick response) code. The machine-readable optical label may be configured to identify the device. An electronic reader capable of reading machine-readable optical labels may be used to read and record the result. An electronic reader may be, for example, a smart phone, a tablet, a laptop, or bar code reader or QR code reader. The electronic reader may further be used to transmit the result, for example, to a healthcare provider or to a database.

A method of using the sample collection system may include exhaling into the exhalation piece (while the housing 10 is assembled), either through the mouth or through the nose, to capture a sample in the porous sample collection media 300; coupling the housing 10 of the sample collection device 1 with a sample collection tube 20, and transferring the sample into the sample collection tube 20. The sample may be transferred by dislodging the sample collection media 300 and pushing the sample collection media 300 into the sample collection tube 20. The sample collection media 300 may be dislodged and pushed using a plunger 30.

Optionally, the method may include applying a liquid to the porous sample collection media 300. The liquid may be applied in an amount suitable for eluting viruses, pathogens, or other analytes, captured in the porous sample collection media. A suitable amount of liquid may be determined as a ratio of liquid volume to the surface area of the porous sample collection media. For example, the volume of liquid may be in a range from 10 μm/cm² to 400 μm/cm², or from 10 μm/cm² to 250 μm/cm², or from 50 μm/cm² to 150 μm/cm². In some embodiments, the volume of liquid is from 50 μm to 500 μm.

Optionally, a user may cap either the sample collection device 1 or the sample collection tube 20 or both after obtaining a sample and transferring the sample to the sample collection tube 20. The sample collection device 1 may be capped for storage or safe disposal. The sample collection tube 20 may be capped for analysis or transport.

The method may further include vortexing the sample collection tube 20 containing the loaded sample collection media 30.

In another configuration, a kit may include the sample collection device and instructions for collecting a sample onto the sample collection media and transferring the sample collection media into a sample collection tube. The instructions may include instructions to: exhale along the airflow path to capture a sample in the porous sample collection media; couple the housing with a sample collection tube; and use a plunger to push the sample collection media into a sample collection tube coupled with the device. The instructions may further include instructions to read an optical label using an electronic reader.

All references and publications cited herein are expressly incorporated herein by reference in their entirety into this disclosure, except to the extent they may directly contradict this disclosure. Although specific embodiments have been illustrated and described herein, it will be appreciated by those of ordinary skill in the art that a variety of alternate and/or equivalent implementations can be substituted for the specific embodiments shown and described without departing from the scope of the present disclosure. It should be understood that this disclosure is not intended to be unduly limited by the illustrative embodiments and examples set forth herein and that such examples and embodiments are presented by way of example only with the scope of the disclosure intended to be limited only by the claims set forth here.

1. A sample collection device comprising:
a housing comprising a first part and a second part removably coupled with one another, and an airflow path extending through the first and second parts, the first part comprising an exhalation piece and the second part comprising a coupling end constructed for coupling with a sample collection tube; and
porous sample collection media comprising nonwoven material, partially disposed between the first and second parts and arranged to occlude the airflow path
wherein the second part comprises a coupling mechanism constructed for coupling with a sample collection tube, the coupling mechanism comprising either:
a first set of threads; or
a frustoconical portion and a plurality of ribs extending from the frustoconical portion.

2. The sample collection device of claim 1, wherein each of the first part and the second part comprise a tubular body.

3. The sample collection device of claim 1, wherein the tubular body of the first part is coaxial with the tubular body of the second part when the first and second parts are coupled.

4. The sample collection device of claim 1, wherein the second part comprises a ledge and the first part comprises a distal end, and wherein an outer edge of the porous sample collection media is captured between the ledge and the distal end when the first and second parts are coupled.

5. (canceled)

6. The sample collection device of claim 5, wherein the nonwoven material comprises polylactic acid, polypropylene, or a combination thereof.

7. The sample collection device of claim 1, wherein the first and second parts are coupled by bayonet coupling, interference fit, snap fit, or threaded coupling.

8. The sample collection device of claim 1, wherein the housing comprises a finger support extending outwardly from a body of the first part or the second part.

9. (canceled)

10. (canceled)

11. The sample collection device of claim 10, wherein the coupling mechanism comprises a second set of threads of a different size than the first set of threads.

12. The sample collection device of claim 9, wherein the coupling mechanism comprises an angled coupling surface.

13. (canceled)

14. The sample collection device of claim 13, wherein the plurality of radial ribs comprises radial ribs.

15. The sample collection device of claim 14, wherein the radial ribs comprise a first rib adjacent the coupling end and having a first diameter, and a second rib having a second diameter that is greater than the first diameter.

16. The sample collection device of claim 15, wherein the radial ribs further comprise a third rib between the first rib and second rib, the third rib and the first rib forming a pair of ribs each having the first diameter.

17. The sample collection device of claim 2, wherein the first part comprises a flange extending outwardly from a proximal end of the tubular body.

18. The sample collection device of claim 17, wherein the flange comprises a cut-away.

19. (canceled)

20. A system for collecting a sample from breath comprising the sample collection device of claim 1 and a plunger for releasing the porous sample collection media from the housing.

21. The system of claim 20 further comprising a sample collection tube capable of coupling with the second part and/or the coupling portion.

22. The system of claim 20, wherein the system is constructed to release the porous sample collection media into the sample collection tube coupled with the sample collection device upon pushing with the plunger.

23. The system of claim 20, further comprising a seal between (1) the second part or the coupling portion and (2) the sample collection tube.

24. A method of obtaining a sample using a sample collection device comprising:
providing the sample collection device of claim;
breathing into the exhalation piece or portion to collect a sample on the porous sample collection media;
coupling the housing with a sample collection tube; and
transferring the collected sample on the porous sample collection media into the sample collection tube, wherein the transferring is done by pushing with a plunger.

25. (canceled)

26. The method of claim 24, wherein the transferring is done by applying a liquid onto the porous sample collection media.

27-29. (canceled)
